(19) United States
(12) Patent Application Publication
(10) Pub. No.: US 2021/0338787 A1
(43) Pub. Date: Nov. 4, 2021

(54) METHODS FOR TREATING CORONAVIRUS INFECTION

(71) Applicant: CHILDREN'S HOSPITAL MEDICAL CENTER, Cincinnati, OH (US)

(72) Inventors: Marc E. Rothenberg, Cincinnati, OH (US); Nurit P. Azouz, Cincinnati, OH (US)

(21) Appl. No.: 17/302,247

(22) Filed: Apr. 28, 2021

Related U.S. Application Data
(60) Provisional application No. 63/017,027, filed on Apr. 29, 2020.

(51) Int. Cl. A61K 38/57; A61K 31/245; A61P 31/14; G01N 33/50; G01N 33/573

(52) U.S. Cl. CPC A61K 38/57; A61K 31/245; G01N 33/573; G01N 33/502; A61P 31/14

(57) ABSTRACT

The present invention relates to methods for treating or preventing a coronavirus infection with serine protease inhibitors targeted against the host protease, transmembrane serine protease 2 (TMPRSS2), and related compositions and methods.

### CROSS REFERENCE TO RELATED APPLICATIONS

This application claims the benefit of U.S. Provisional Application No. 63/017,027 filed Apr. 29, 2020, the disclosure of which is incorporated herein by reference in its entirety.

### STATEMENT REGARDING FEDERALLY-SPONSORED RESEARCH

This invention was made with government support under U19 A1070235 awarded by National Institutes of Health. The government has certain rights in the invention.

### FIELD OF THE INVENTION

The invention disclosed herein relates to therapy for coronavirus infection and related assays and methods.

### BACKGROUND OF THE INVENTION

The COVID-19 pandemic is caused by the severe acute respiratory syndrome (SARS)-coronavirus (CoV) 2. The efficient transmission of this virus has led to exponential growth in the number of worldwide cases. Similar to other coronaviruses, SARS-CoV-2 entry into host cells relies on the proteolytic processing of spike (S) protein by host proteases and engagement of the angiotensin-converting enzyme 2 (ACE2) receptor (Hoffmann et al., Cell 2020; 181(2):271-80 e8). Several proteases are crucial to coronavirus viral entry, and they are found at different subcellular locations. The S protein cleavage may occur extracellularly near the plasma membrane by cell surface proteases or intracellularly by lysosomal endopeptidase enzymes, such as cathepsin L, which facilitate viral entry by activating membrane fusion and subsequent cell entry through endocytosis, as in the case of MERS-CoV (Qing et al., Methods Mol Biol. 2020; 2099:9-20). Despite intensive research of the SARS-CoV-2 life cycle, the cellular location of the SARS-CoV-2 S protein priming remains debatable. In particular, the interplay between the extracellular and intracellular proteases in the membrane fusion and cell entry of SARS-CoV-2 is controversial.

Transmembrane serine protease 2 (TMPRSS2), a cell surface serine protease, may be involved in cell entry of SARS-CoV-2. TMPRSS2 has been shown to cleave ACE2 at arginine and lysine residues within ACE2 amino acids 697-716, which enhances cell entry (Heurich et al., Protein. J Virol. 2014; 88(2):1293-307). TMPRSS2 increases the entry of another coronavirus SARS-CoV, not only by processing of the S protein, but also by processing of the host receptor ACE2 (Heurich et al., J Virol. 2014; 88(2):1293-307). Consistent with these findings, TMPRSS2-deficient mice have decreased viral spread of

SARS-CoV in the airways compared to that of control mice. In addition, the drug camostat mesylate (camostat), which inhibits a number of proteases including TMPRSS2, was shown to inhibit SARS-CoV-2 entry into cells in vitro (Matsuyama et al., Proc Natl Acad Sci USA. 2020; 117(13):7001-3). Therefore, TMPRSS2 is regarded as one of the most important proteases for S protein priming and cell entry of SARS-CoV-2.

There remains a need to identify new TMPRSS2 inhibitors for treating coronavirus infections. The present invention addresses this need.

### SUMMARY OF THE INVENTION

The disclosure provides methods for treating or preventing a coronavirus infection in a subject in need thereof, the methods comprising administering to the subject a pharmaceutical composition comprising a serine protease inhibitor which irreversibly inhibits serine proteases and cannot diffuse across a phospholipid bilayer of a mammalian cell membrane. In embodiments, the serine protease inhibitor is an irreversible inhibitor of transmembrane serine protease 2 (TMPRSS2). In embodiments, the serine protease inhibitor is alpha 1 antitrypsin (A1AT). In embodiments, the methods may further comprise administering a second serine protease inhibitor to the subject. In embodiments, the second serine protease is camostat, or a pharmaceutically acceptable salt thereof. In accordance with any of the foregoing embodiments, the subject may be a mammal, preferably a human. In accordance with any of the foregoing embodiments, the subject in need may be one who has tested positive for a coronavirus infection; one who has been in close contact with one or more persons who have tested positive for a coronavirus infection; or one who is deemed to be at risk of contracting a coronavirus infection.

In accordance with any of the foregoing embodiments, the coronavirus may be selected from MERS-CoV, SARS-CoV, and SARS-CoV-2. In embodiments, the coronavirus is SARS-CoV-2.

In an embodiment, the disclosure provides a method for treating or preventing a SARS-CoV-2 infection in a human subject in need thereof, the method comprising administering to the subject a pharmaceutical composition comprising alpha 1 antitrypsin (A1AT). In embodiments, the method further comprises administering a second serine protease inhibitor to the subject. In embodiments, the second serine protease is camostat, or a pharmaceutically acceptable salt thereof. In embodiments, the subject in need is one who has tested positive for a coronavirus infection; one who has been in close contact with one or more persons who have tested positive for a coronavirus infection; or one who is deemed to be at risk of contracting a coronavirus infection.

The disclosure also provides methods for reducing cell to cell spread of a coronavirus in a subject in need thereof, the methods comprising administering to the subject a composition comprising a serine protease inhibitor which irreversibly inhibits serine proteases and cannot diffuse across a phospholipid bilayer of a mammalian cell membrane.

The disclosure also provides methods for inhibiting dissemination of a coronavirus in a population of subjects, the methods comprising administering to a plurality of subjects in the population a composition comprising a serine protease inhibitor which irreversibly inhibits serine proteases and cannot diffuse across a phospholipid bilayer of a mammalian cell membrane.

In accordance with either of the two foregoing embodiments, the serine protease inhibitor is an irreversible inhibitor of transmembrane serine protease 2 (TMPRSS2). In embodiments, the serine protease inhibitor is alpha 1 antitrypsin (A1AT).

The disclosure also provides methods for identifying an inhibitor of transmembrane serine protease 2 (TMPRSS2), the methods comprising contacting in vitro or ex vivo a plurality of recombinant cells with the test compound in the presence of a TMPRSS2 substrate, wherein the recombinant cells overexpress a proteolytically active recombinant transmembrane serine protease 2 (TMPRSS2), optionally comprising a peptide or protein tag element, and detecting cleavage of the TMPRSS2 substrate, wherein reduced cleavage of the TMPRSS2 substrate in the presence of the test compound indicates the test compound is an inhibitor of TMPRSS2. In embodiments, the TMPRSS2 substrate is a flurogenic or colormetric substrate and cleavage is detected by detecting fluorescence or color. In embodiments, the substrate is Boc-Gln-Ala-Arg-7-Amino-4-methylcoumarin (BOC-QAR-AMC). In embodiments, the tag element is selected from a histidine tag, a FLAG tag, a human influenza hemagglutinin (HA) tag, a Myc tag, and a V5 tag. In embodiments, the tag element is selected from a green fluorescent protein (GFP), GST glutathione-S-transferase (GST), β-galactosidase (β-GAL), luciferase, maltose binding protein (MBP), calmodulin binding protein (CBP), red fluorescence protein (RFP), and vesicular stomatitis virus glycoprotein (VSV-G).

### BRIEF DESCRIPTION OF THE FIGURES

FIG. 1A-D: Measurements of TMPRSS2 activity in transfected cells. A. Western blot of TMPRSS2 protein expression in HEK-293T cells transfected with PLX304 vector or PLX304-TMPRSS2 vector is shown. TMPRSS2 containing a C terminal V5 (TMPRSS2-V5) tag was assessed by anti-VS antibody, and anti-GAPDH antibody was used as a loading control. B. Arbitrary fluorescence unit (AFU) measurements of control or TMPRSS-overexpressing cells incubated with BOC-QAR-AMC for 75 minutes at 37° C. are shown. Wells containing PBS and BOC-QAR-AMC were used as background fluorescence reads. C. Fluorescence of control or TMPRSS2-overexpressing cells was measured every 15 minutes for a total time of 180 minutes. D. The average proteolytic activity rate per minute of control or TMPRSS2-overexpressing cells. The fluorescent signal was measured by the UV filter (excitation 365 nm and emission 410 nm). Data in B, D, and E represent the mean±SD with interquartile ranges in B and D. GAPDH, glyceraldehyde-3-phosphate dehydrogenase; TMPRSS2, transmembrane serine protease 2.

FIG. 2A-C: The effect of intracellular and extracellular inhibitors on TMPRSS2 activity. Fluorescence of TMPRSS2-overexpressing cells was measured every 15 minutes in the presence of the indicated concentrations of camostat mesylate (camostat) (A), secretory leukocyte protease inhibitor (SLPI) (B), or A1AT (C). The results in A-C are presented as the means±SE from at least 3 independent experiments performed in duplicate and/or triplicate. TMPRSS2, transmembrane serine protease 2.

FIG. 3A-C: Schematic inhibition of TMPRSS2 by A1AT. A. General inhibitory mechanism of serpins applied to TMPRSS2 and A1AT are shown. The homology model of TMPRSS2 is shown as the blue cartoon and A1AT as the grey cartoon, with the reactive center loop highlighted in gold. B. Shown are the interactions at the interface of the Michaelis complex model, highlighting LYS340 and LYS390 of TMPRSS2 (blue) and GLU199, ASP202, and ASP260 of A1AT (grey). C. A close-up of the Michaelis complex at the active site region is shown. The catalytic triad residues HIS296, ASP345, and SER441 are depicted in black. Relevant residues are represented as sticks, hydrogen bonds are represented as dashed black lines, and the cleavage site is indicated by a black arrow. Note that there are hydrogen bonds at the oxyanion hole between GLY439/SER441 of TMPRSS2 and MET358 of A1AT. A1AT, alpha 1 antitrypsin; TMPRSS2, transmembrane serine protease 2.

FIG. 4A-B: The effect of A1AT and camostat on SARS-CoV-2 infection. A. Intracellular SARS-CoV-2 genomic copies were analyzed twenty hours after infection of Caco-2 cells in the presence of A1AT (10 μM), camostat (10 μM), or control media. Data represent fold change in CoV2 copy number compared to control media. Each data point represents 1 well with the mean±SD of 3 independent experiments. B. SARS-CoV-2 virus production was analyzed 24 and 48 hours after infection of Calu-3 cells at the indicated concentration of A1AT and camostat, and in control media. Data represent fold change in plaque number compared to control media. Results are the mean±SE of 2 or more independent experiments. A1AT, alpha 1 antitrypsin.

FIG. 5A-G: A1AT concentration in plasma samples of patients with COVID-19 and suggested role in SARS-CoV-2 cell entry. A1AT plasma concentrations in patients who were positive for COVID-19 are shown and stratified according to disease severity at the time of disposition from the Emergency Department (A) or the maximal severity within 30 days of index Emergency Department visit (Max severity) (B) (1=Outpatient, 2=Hospitalized, 3=Intensive Care Unit or Death). In (C), correlation between plasma A1AT concentrations and plasma IL-10, IL-6, IL-8, or TNFα concentrations, r and P values were calculated according to Spearman correlation. D. Plasma concentration of A1AT and IL-6 in each patient with confirmed COVID-19 with markers representing individual patients. Ratio of IL6/A1AT plasma concentrations in patients who were positive for COVID-19 are shown and stratified according to disease severity at the time of disposition from the Emergency Department (E) or the maximal severity within 30 days of index Emergency Department visit (Max severity) (F). G. Model of SARS-CoV-2 entry mediated by extracellular proteolytic events. Extracellular proteases, such as TMPRSS2, process the S protein on the SARS-CoV-2 envelope in a process called priming. Priming of the S protein is necessary for binding between the S protein and the host receptor angiotensin-converting enzyme 2 (ACE2). Extracellular inhibitors, such as A1AT, prevent the priming of the S protein and inhibit virus entry. In addition, inhibiting transmembrane serine protease 2 (TMPRSS2) prevents processing of ACE2, which decreases the infectivity of the coronavirus. Model was illustrated using Biorender. A1AT, alpha 1 antitrypsin; IL, interleukin.

### DETAILED DESCRIPTION OF THE INVENTION

The present invention is based, in part, on the development of a cell-based assay to identify TMPRSS2 inhibitors, the identification of alpha 1 antitrypsin (A1AT) as an inhibitor of TMPRSS2, evidence signifying the importance of extracellular proteases for coronavirus entry into mammalian cells, and evidence demonstrating that A1AT not only inhibits TMPRSS2 but also inhibits SARS-CoV-2 infection.

Accordingly, the disclosure provides methods for treating or preventing a coronavirus infection in a subject in need thereof, the methods comprising administering to the subject a pharmaceutical composition comprising a serine protease inhibitor which irreversibly inhibits serine proteases and cannot diffuse across a phospholipid bilayer of a mammalian cell membrane. In embodiments, the serine protease inhibitor is an irreversible inhibitor of transmembrane serine protease 2 (TMPRSS2). In a preferred embodiment, the serine protease inhibitor is alpha 1 antitrypsin, referred to herein as A1AT, also referred to as AAT in some studies. The disclosure also provides related methods for reducing cell to cell spread of a coronavirus in a mammal and inhibiting dissemination of a coronavirus in a population, the methods comprising administering to the mammal or a plurality of subjects in the population a serine protease inhibitor which irreversibly inhibits serine proteases and cannot diffuse across a phospholipid bilayer of a mammalian cell membrane.

In embodiments, the disclosure provides methods of treating a coronavirus infection wherein the coronavirus is SARS-CoV-2 in a subject in need thereof, the methods comprising administering to the subject a pharmaceutical composition comprising a serine protease inhibitor which irreversibly inhibits serine proteases and cannot diffuse across a phospholipid bilayer of a mammalian cell membrane. In embodiments, the serine protease inhibitor is an irreversible inhibitor of transmembrane serine protease 2 (TMPRSS2). In a preferred embodiment, the serine protease inhibitor is A1AT. In embodiments, the methods of treating include slowing or preventing disease progression. In the context of SARS-CoV-2 infection, the disease, i.e., COVID-19, may generally be described as having at least three distinct phases or stages. In the first stage, which corresponds to the initial host immune repsonse to the virus, the subject generally presents with symptoms consistent with an upper respiratory tract infection such as cough, congestion, runny nose, sore throat; but other symptoms may predominate including fever or chills, muscle or body aches, headache, fatigue, etc. In stage two, the subject may present with more severe pulmonary symptoms, including for example shortness or breath or difficulty breathing and pneumonia. In stage three, the subject presents with systems of hyperinflammation and may for example develop acute respiratory distress syndrome (ARDS), sepsis, kidney failure, and other organ failures. Accordingly, in embodiments, the methods described here prevent the progression of COVID-19 from stage 1 to stage 2, or from stage 2 to stage 3.

The present disclosure also provides methods comprising combination therapy. As used herein, “combination therapy” or “co-therapy” includes the administration of at least one additional therapy, as part of a treatment regimen intended to provide a beneficial effect from the co-action of the irreversible inhibitor of TMPRSS2 and the additional therapy. The terms “combination therapy” or “combination therapy regimen” are not intended to encompass the administration of two or more therapies as part of separate monotherapy regimens that incidentally and arbitrarily result in a beneficial effect that was not intended or predicted. Preferably, the administration of a combination therapy as discussed here provides a synergistic response in the subject being treated. In this context, the term “synergistic” refers to the efficacy of the combination being more than the additive effects of either single therapy alone. The synergistic effect of a combination therapy according to the disclosure may provide a number of beneficial effects including providing for the use of lower dosages and/or less frequent administration of at least one therapy in the combination compared to its dose and/or frequency outside of the combination. Additional beneficial effects of the combination can be manifested in the avoidance or reduction of adverse or unwanted side effects associated with the use of either therapy in the combination alone (also referred to as monotherapy). In embodiments, the methods of combination therapy for treating a coronavirus infection, may comprise administering an irreversible inhibitor of TMPRSS2 and at least one additional therapy such as an additional serine protease inhibitor. In embodiments, the additional serine protease inhibitor is camostat, or a pharmaceutically acceptable salt thereof.

The disclosure also provides cell-based assays for identifying TMPRSS2 inhibitors. In embodiments, the assay method comprises contacting in vitro or ex vivo a plurality of recombinant cells with a test compound in the presence of a TMPRSS2 substrate, wherein the recombinant cells overexpress a proteolytically active recombinant transmembrane serine protease 2 (TMPRSS2) comprising a peptide or protein tag element, and detecting cleavage of the TMPRSS2 substrate, wherein reduced cleavage of the TMPRSS2 substrate in the presence of the test compound indicates the test compound is an inhibitor of TMPRSS2.

In the context of the assay methods described here, recombinant cells are considered to “overexpress” an enzymatically active protein, such as TMPRSS2, where the recombinant cells show at least about a 2-fold increase in enzymatic activity of the protein compared to non-recombinant cells, e.g., compared to the same type of cells not carrying the recombinant protein.

In the context of the assay methods described here, the recombinant cells express a recombinant protein, namely a TMPRSS2 protein, which has been introduced into the cells, e.g., by transfection of an expression vector encoding the protein, e.g., a bacterial plasmid-derived expression vector or viral expression vector such as a lentiviral vector. In embodiments, the TMPRSS2 sequence used to construct the expression vector is a human TMPRSS2, for example the sequence defined by NCBI Reference Sequence NM_ 001135099.1, NM_001382720.1, NM_005656.4, and related sequences, e.g., additional isoforms. Typically, the transfected protein will further comprise an element, or “tag” fused to either its C- or N-terminus, which may be referred to herein as a “tag element”. In this context, a tag element is a heterologous peptide or protein sequence which when fused to the transfected protein, e.g., TMPRSS2, facilitates its purification and/or detection. Any tag element may be used in this context. Exemplary tag elements in the form of short peptides include a polyhistidine tag, which generally comprises 6-8 histidine residues; a FLAG tag; a human influenza hemagglutinin (HA) tag, which is a peptide sequence derived from the surface glycoprotein of the influenza virus; a Myc tag, which is a peptide derived from the c-myc gene product; and a V5 tag, which is derived from a small epitope (Pk) found on the P and V proteins of the paramyxovirus. Exemplary tag elements in the form of proteins include a green fluorescent protein (GFP), GST glutathione-S-transferase (GST), β-galactosidase (β-GAL), luciferase, maltose binding protein (MBP), calmodulin binding protein (CBP), red fluorescence protein (RFP), and vesicular stomatitis virus glycoprotein (VSV-G).

The methods described here are generally applicable to human subjects, also referred to as “patients”, but the methods may be applied to other mammalian subjects. Accordingly, in embodiments a method described here may be performed on a “subject” which may include any mammal, for example a human, non-human primate, mouse, rat, dog, cat, cow, horse, goat, camel, sheep or a pig. Preferably, the subject is a human. The term “patient” refers to a human subject.

In accordance with the methods described here a subject “in need of” treatment may be a subject who has tested positive for a coronavirus infection but does not present with clinical symptoms of coronavirus infection; one who has been in close contact with one or more persons who have tested positive for a coronavirus infection; or one who is deemed to be at risk of contracting a coronavirus infection. In embodiments, the subject “in need of” treatment may be a subject who diagnosed with COVID-19 based on clinical symptoms of COVID-19 and a positive test for SARS-CoV-2.

In accordance with the methods described here, in some embodiments, a subject “in need of” treatment is a subject diagnosed with COVID-19; a subject at risk of developing COVID-19; a subject who has tested positive for SARS-CoV-2; or a subject at risk of exposure to SARS-CoV-2.

The terms “determining,” “measuring,” and “assaying” are used interchangeably herein and can include quantitative and/or qualitative determinations. These terms are intended to exclude purely mental steps and instead refer to the use of one or more laboratory assays for determining the presence or quantity of an analyte in a sample. Such methods may include computer assisted steps for determining the amount of an analyte, such as the amount of a protein, peptide or polypeptide, lipid, polysaccharide, antibody or fragment thereof, immunoglobulin molecule, nucleic acid, such as RNA or DNA, etc.

In the context of the present disclosure, the terms “treatment”, “treating”, or “treat” describe the management and care of a subject for the purpose of combating a disease or disorder, such as a coronavirus infection, and may include the administration of a therapeutic agent as well as the administration of other therapies to alleviate one or more symptoms or complications of coronavirus infection, thereby treating the coronavirus infection. Thus, in the context of the methods described here, the term “treating” may refer to the amelioration or stabilization of one or more symptoms associated with a coronavirus infection. The term “treating” may also refer to preventing disease progression.

As used herein, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

As used herein, the term “about,” as used herein when referring to a measurable value such as an amount of a compound or agent of this invention, dose, time, temperature, and the like, is meant to encompass variations likely to be less than±5-10% of the specified amount.

As used herein, the transitional phrase “consisting essentially of” is to be interpreted as encompassing the recited materials or steps and those that do not materially affect the basic and novel characteristic(s) of the claimed invention. This term is not equivalent to the open-ended term “comprising.”

### EXAMPLES

As described in more detail below, the efficiency of TMPRSS2 inhibition by synthetic and natural serine protease inhibitors that are cell permeable or have extracellular function was compared and alpha 1 antitrypsin (A1AT) identified as a novel inhibitor of TMPRSS2. Structural modeling of the Michaelis complex between TMPRSS2 and A1AT indicated that they dock on the cell surface in a conformation is suitable for catalysis, resembling similar serine protease-inhibitor complexes. Further provided is evidence for the utility of A1AT as an antiviral agent in SARS-CoV-2 infection. A1AT decreased SARS-CoV-2 copy number within target cells when applied during infection. The effect of A1AT was comparable to the effect of camostat, which was previously shown to inhibit SARS-CoV-2 cell entry. In contrast to camostat, which is a cell permeable drug, A1AT is a circulating extracellular protein that inhibits extracellular proteases and does not possess access to intracellular proteases. These findings emphasize the importance of extracellular proteases to viral cell entry. By inhibiting extracellular proteolytic activity, A1AT can potentially inhibit S protein processing and limit SARS-CoV-2 cell-cell spread and dissemination.

### Overexpressing TMPRSS2 and Measuring Proteolytic Activity

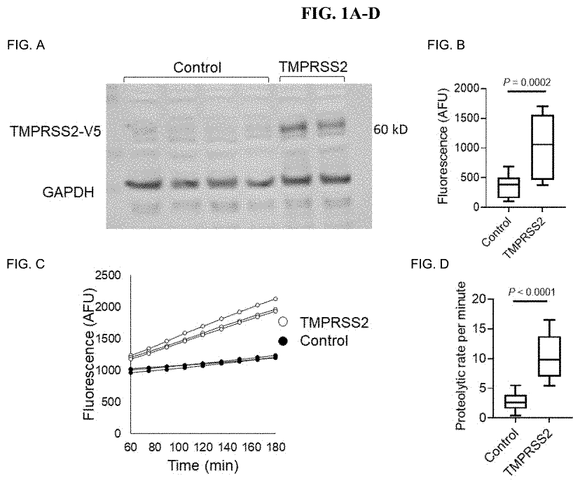

The following describes the development of an experimental framework for quantifying TMPRSS2 proteolytic activity. TMPRSS2 with a C-terminal V5 tag was overexpressed in a human cell line, HEK-293T, because of its high transfectability. Western blot analysis of the cell lysates revealed a band at ˜60 kD in TMPRSS2-transfected cells but not in control cells (FIG. 1A). GAPDH was used as a loading control. Measurements of the proteolytic activity of the transfected cells using the fluorogenic peptide substrate Boc-Gln-Ala-Arg-7-Amino-4-methylcoumarin (BOC-QAR-AMC) revealed a >2.5-fold increase in the proteolytic activity of the TMPRSS2-transfected cells compared with that of control cells (P=0.0002; FIG. 1B). The proteolytic activity of the TMPRSS2-transfected cells was increased compared with that of control cells (FIG. 1C). The mean proteolytic rate per minute of the TMPRSS2-transfected cells was increased by >3.5 fold compared to the proteolytic rate of control cells (P<0.0001, FIG. 1D). Using serial dilutions of recombinant TMPRSS2, we estimated that the amount of TMPRSS2 that is expressed by TMPRSS2-overexpressing cells is about 100 ng/well (Supplementary FIG. 1). These collective data demonstrated that overexpression of TMPRSS2 resulted in overproduction of functional TMPRSS2 and established an experimental system for accurately measuring the proteolytic activity of TMPRSS2.

### Identifying Functional TMPRSS2 Inhibitors

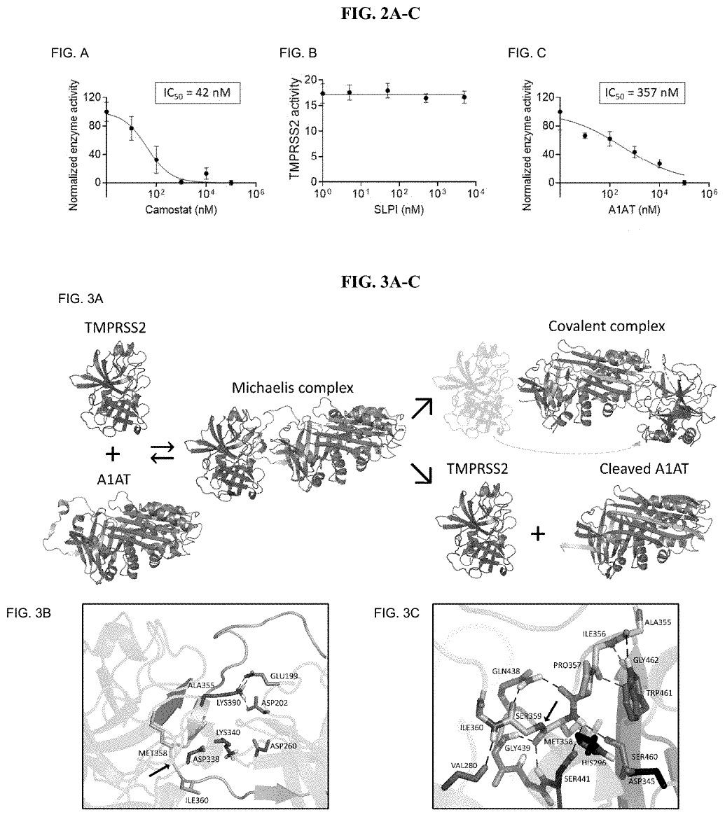

The effect of protease inhibitors on TMPRSS2 activity was tested. As a positive control, cells were treated with camostat mesylate, a drug that has been shown to inhibit TMPRSS2. As expected, camostat mesylate inhibited the proteolytic activity of TMPRSS2 with a calculated IC50 of 42 nM (FIG. 2A). We then tested whether the secretory leukocyte protease inhibitor (SLPI) would inhibit TMPRSS2. However, none of the tested concentrations of SLPI inhibited TMPRSS2 proteolytic activity (FIG. 2B). In contrast, A1AT inhibited TMPRSS2 proteolytic activity in a dose-dependent manner (IC50 of 357 nM; FIG. 2C). A1AT did not demonstrate toxic effects in the tested concentrations as demonstrated by viability assays of HEK-293T cells (data not shown).

### Modeling the Eextracellular TMPRSS2-A1AT Michaelis Complex

The Michaelis complex between TMPRSS2 and A1AT was modeled to better understand the structural basis of TMPRSS2 inhibition prior to A1AT cleavage and covalent attachment (FIG. 3A). The results suggest that TMPRSS2 interacts with A1AT through its reactive center loop (RCL), driven by complementary electrostatic interactions at their surfaces (FIG. 3B). Namely, LYS390 (TMPRSS2) forms a strong bifurcated salt bridge with GLU199/ASP202 (A1AT), while LYS340 and ASP260 form a second electrostatic contact, whose proximity may be limited by the presence of ASP338 at the surface of TMPRSS2. In the active site region, TMPRSS2 interacts with A1AT via an extensive hydrogen bond network (FIG. 3C). Part of these interactions stabilize a short, antiparallel beta-sheet between GLY462 and ILE356-PRO357, similar to the one present in 1 OPH of the Research Collaboratory for Structural Bioinformatics (RCSB) Protein Data Bank (PDB). At the entrance of the S1 pocket, GLN438 forms hydrogen bonds with PRO357 and SER359, fixing and orienting the A1AT backbone around the reactive peptide bond (MET358-SER359). As a result, MET358 is buried inside the S1 pocket, with its backbone carbonyl placed inside the oxyanion hole, forming hydrogen bonds with the GLY439 and SER441 (TMPRSS2) backbone amides. These predicted interactions, together with the overall orientation of the RCL and the catalytic triad, constitute a suitable environment for the cleavage and covalent binding of A1AT to TMPRSS2.

### Analyzing the A1AT Effect on TMPRSS2-Mediated SARS-CoV-2 Infectivity

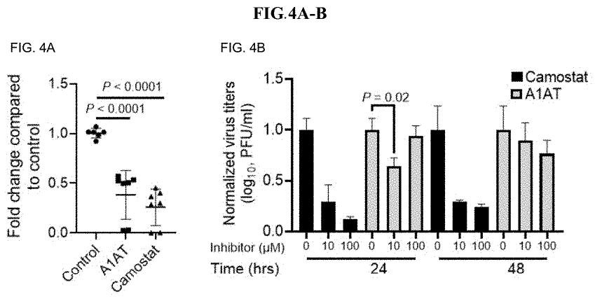

The ability of A1AT to inhibit SARS-CoV-2 infectivity was investigated in cells that are commonly used for SARS-CoV-2 assays because of their TMPRSS2 expression. Caco-2 cells were either left untreated or treated with either A1AT (10 μM) or camostat (10 μM); cells were then infected with SARS-CoV-2. Twenty hours later, quantifying the genomic SARS-CoV-2 from the intracellular RNA revealed a significant decrease in the viral load in cells that were treated with A1AT or camostat (−2.6 and −3.9 fold, respectively) compared with untreated control cells (P<0.0001 for both inhibitors; FIG. 4A). The effect of A1AT and camostat on the infectivity of SARS-CoV-2 in Calu-3 cells was tested next. Both inhibitors decreased SARS-CoV-2 plaque formation when analyzed 24 hours after infection (FIG. 4B). A1AT did not decrease plaque formation 48 hours after infection while the effect of camostat lasted on that time point (FIG. 4B). These data suggest that A1AT can limit the SARS-CoV-2 life cycle by modulating TMPRSS2 activity in the host cells. Importantly, A1AT and camostat were nontoxic in Caco-2 cells in the tested concentrations (data not shown).

### Plasma A1AT Levels in Patients With COVID-19

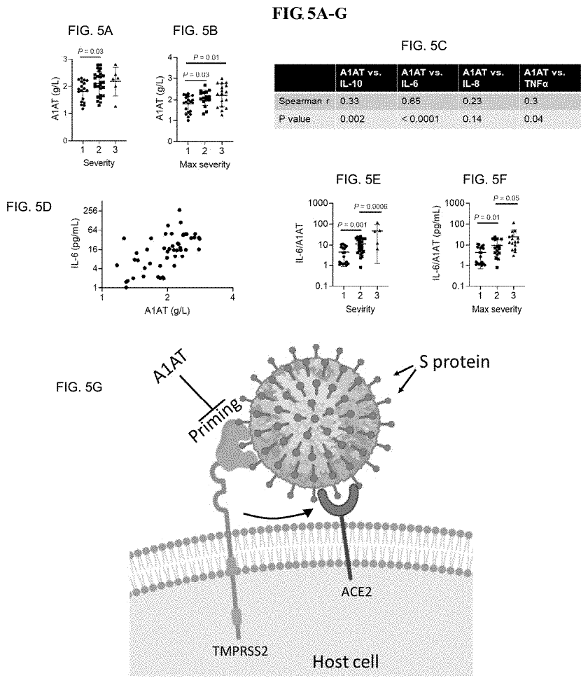

A1AT is normally found at high concentrations in the blood and increases during acute phase responses or tissue injury. We hypothesized that A1AT concentrations in plasma samples from patients with COVID-19 would correlate with disease severity as part of the anti-SARS-CoV-2 response. To test this hypothesis, we analyzed plasma A1AT levels in a cohort of patients who tested positive for COVID-19. These patients were divided into 3 groups according to disease severity at the time of emergency department disposition and the maximal severity within 30 days (1 mild—outpatient care, 2 moderate—need for hospitalization, 3 severe—need for intensive care unit admission; see methods section). A1AT levels were significantly different between the group of patients with mild disease and the group of patients with moderate disease. The mean concentration of A1AT was the highest in the group of patients with severe disease compared to the other groups (FIG. 5A). A1AT concentrations positively correlated with maximal severity of disease (FIG. 5B). Plasma A1AT concentrations correlated with plasma IL-6 (r=0.65, P<0.0001), IL-10 (r=0.33, P=0.002), and TNFα concentrations (r=0.3; P=0.002) but not plasma IL-8 concentrations (FIG. 5C,D). Consistent with previous studies (McElvaney et al Am J Respir Crit Care Med. 2020; 202(6):812-21), we observed that the IL6/A1AT ratio positively correlated with disease severity (FIG. 5E,F)

### Discussion

As discussed above, we developed a cell-based methodology that allows the quantification of TMPRSS2 activity. This methodology enables testing the effect of intracellular compounds and extracellular compounds, thus permitting differentiation between inhibition of intracellular and extracellular protease. Using this methodology, we revealed that A1AT, which is approved by the FDA for the treatment of A1AT deficiency, can efficiently inhibit TMPRSS2. Structural modeling of the A1AT-TMPRSS2 Michaelis complex revealed that A1AT is likely cleaved and covalently bound to TMPRSS2. The importance of A1AT in fighting coronavirus infection is supported by the finding that plasma A1AT levels correlated with COVID-19 severity and with plasma IL-6 levels. A1AT inhibited SARS-CoV-2 infection at a comparable level to camostat in Caco-2 and Calu-3 cells, cell types that are efficiently infected by SARS-CoV-2. Consistent with our findings, Oguntuyo et al demonstrated that SARS-CoV-2 naive serum exhibits significant inhibition of SARS-CoV-2 entry, and that this inhibition is mostly accounted for by the presence of A1AT in the sera Oguntuyo et al., Pathogenesis and Therapeutics. bioRxiv. August 2020. These data suggest that A1AT treatment may benefit COVID-19 countermeasures by inhibiting extracellular-mediated S protein processing and virus entry. Notably, the inhibitory effect of camostat on SARS-CoV-2 infection in Calu-3 cells persisted longer compared to A1AT which inhibited the infection after 24 hours but not after 48 hours. This difference may stem from a different mechanism of action of these 2 drugs; A1AT inhibits extracellular and membranal proteases, while camostat penetrates the cells and therefore, can potentially inhibit intracellular proteases that affect multiple steps in the SARS-CoV-2 life cycle which therefore prolongs its effect.

Though the relative contribution of intracellular proteases and extracellular proteases to the S protein priming and cell entry of SARS-CoV-2 has yet to be determined, the results presented here provide evidence that extracellular protease activity is rate-limiting in the process of SARS-CoV-2 cell entry. Thus, extracellular protease inhibitors provide a mechanism for inhibiting SARS-CoV-2 entry and cell-to-cell transmission by modulating the exterior of the host cells.

Targeting the host extracellular proteases, such as TMPRSS2, has several advantages over targeting viral proteins. First, anti-virals can rapidly lose their effectiveness due to the high rate of mutations that occur in the viral genome, but targeting host proteins limits the risk of drug-resistant viruses due to the relatively low rate of mutations in the host genome. Notably, the obstacle in targeting human proteins is the potential risk of altering physiologic pathways. It has been suggested that TMPRSS2 initiates a cascade of proteolytic activation events that regulate processing of proteins in seminal fluid and in the lung because TMPRSS2 regulates the sodium channel ENaC. Nevertheless, mice deficient in TMPRSS2 lack any obvious phenotypes, suggesting that other proteases may have redundant roles and may compensate for the loss of TMPRSS2. Therefore, delivery of TMPRSS2 inhibitors during viral infections is likely a relatively safe strategy. Although the safety of TMPRSS2 inhibition has not been clinically proven yet, drugs with proteolytic inhibition activity towards TMPRSS2 (eg, camostat mesylate and nafamostat mesylate) are currently being pursued for the treatment of COVID-19. Notably, unlike extracellular A1AT, camostat and nafamostat are cell permeable and therefore may possess undesired intracellular protease inhibition capacity. Second, inhibiting cell entry is an upstream intervention method that limits the overall viral burden and the spread to and replication within tissues, such as the salivary glands, that have important pathologic consequences involved in viral transmission to others. Third, inhibiting cell entry may prevent several downstream disease outcomes. For example, SARS-CoV-2-infected cells undergo cell death by pyroptosis, a process that is thought to induce complications, such as cytokine storm and intra-vascular thrombosis, and thereby results in severe disease outcomes. Moreover, cell death of key alveolar cells induces tissue damage that progresses into acute respiratory distress syndrome, a severe clinical phenotype of COVID-19. Therefore, inhibiting SARS-CoV-2 infection and cell entry by inhibiting the proteolytic priming of the coronavirus S protein is likely to decrease cell death and thereby reduce disease severity.

To our knowledge, we are the first to demonstrate that A1AT inhibits TMPRSS2, which is an extracellular protease with a key role in the entry of SARS-CoV-2, SARS-CoV, MERS-CoV, and influenza viruses. A1AT belongs to the super family of serine protease inhibitors (SERPIN) that irreversibly inhibit serine and cysteine proteases. Proteases interact with SERPINs as depicted in FIG. 3A, forming a Michaelis complex in which the reactive center loop (RCL) binds the protease active site (modelled for TMPRSS2 and A1AT in this work). Cleavage of the RCL results in the formation of a transient, covalent complex that can either undergo dissociation (lower right pathway in FIG. 3A; cleaved A1AT represented by PDB 7API) or translocation and irreversible inhibition (upper right pathway in FIG. 3A; represented by PDB 2D26 in the absence of a TMPRSS2-A1AT-specific model). One of the fragments of the cleaved RCL is inserted into the central beta-sheet of A1AT in both pathways, whereas the other fragment (36-aa) is released into the solvent. Notably, the 36-aa fragment is produced as a result of proteolytic cleavage by several serine proteases and possesses physiologic functions. The experiments with virus infection in vitro provide a proof of principle that A1AT not only biochemically inhibits TMPRSS2 but also inhibits SARS-CoV-2 infection.

To a first approximation, if endogenous A1AT was solely anti-inflammatory, one may have expected a negative correlation between sera levels of A1AT and COVID-19 severity. However, endogenous A1AT concentration in the blood can be increased by 6-fold as part of the acute phase of inflammation or tissue injury. Finally, while endogenous A1AT positively correlated with COVID-19 severity, it is notable that our findings represent potential therapeutic effects of exogenously administered A1AT.

In summary, our findings suggest that treatment with extracellular protease inhibitors either alone or in combination with other anti-COVID-19 agents may be a useful antiviral strategy to fight COVID-19. These protease inhibitors have the potential to prevent SARS-CoV-2 entry to host cells by inhibiting S protein priming by TMPRSS2 and other extracellular proteases and binding of the virus to ACE2 (FIG. 5G). A1AT may be particularly effective as it has dual capacity, inhibiting TMPRSS2 (and hence viral uptake and subsequent replication) and possessing anti-inflammatory activity (Janciauskiene S, Welte T. Ann Am Thorac Soc. 2016; 13 Suppl 4:S280-8). Using these inhibitors may be therapeutic in conditions in which TMPRSS2 function is pathogenic, such as in several types of coronavirus and influenza infections. A1AT is readily available and has an established safety profile, making it a good candidate for clinical use.

### Equivalents

Those skilled in the art will recognize or be able to ascertain using no more than routine experimentation, many equivalents to the specific embodiments of the invention described herein. Such equivalents are intended to be encompassed by the following claims.

All references cited herein are incorporated herein by reference in their entirety and for all purposes to the same extent as if each individual publication or patent or patent application was specifically and individually indicated to be incorporated by reference in its entirety for all purposes.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

1. A method for treating or preventing a coronavirus infection in a subject in need thereof, the method comprising administering to the subject a pharmaceutical composition comprising a serine protease inhibitor which irreversibly inhibits serine proteases and cannot diffuse across a phospholipid bilayer of a mammalian cell membrane.

2. The method of claim 1, wherein the serine protease inhibitor is an irreversible inhibitor of transmembrane serine protease 2 (TMPRSS2).

3. The method of claim 1, wherein the serine protease inhibitor is alpha 1 antitrypsin (A1AT).

4. The method of claim 1, further comprising administering a second serine protease inhibitor to the subject.

5. The method of claim 4, wherein the second serine protease is camostat, or a pharmaceutically acceptable salt thereof.

6. The method of claim 1, wherein the subject is a mammal, preferably a human.

7. The method of claim 6, wherein the subject in need is one who has tested positive for a coronavirus infection.

8. The method of claim 6, wherein the subject in need is one who has been in close contact with one or more persons who have tested positive for a coronavirus infection.

9. The method of claim 6, wherein the subject in need is one who is deemed to be at risk of contracting a coronavirus infection.

10. The method of claim 1, wherein the coronavirus is selected from MERS-CoV, SARS-CoV, and SARS-CoV-2.

11. The method of claim 10, wherein the coronavirus is SARS-CoV-2.

12. A method for treating or preventing a SARS-CoV-2 infection in a human subject in need thereof, the method comprising administering to the subject a pharmaceutical composition comprising alpha 1 antitrypsin (A1AT).

13. The method of claim 12, further comprising administering a second serine protease inhibitor to the subject.

14. The method of claim 13, wherein the second serine protease is camostat, or a pharmaceutically acceptable salt thereof.

15. The method of claim 12, wherein the subject in need is one who has tested positive for a coronavirus infection but does not present with clinical symptoms; one who has been in close contact with one or more persons who have tested positive for a coronavirus infection; or one who is deemed to be at risk of contracting a coronavirus infection.

16. The method of claim 15, wherein the coronavirus is SARS-CoV-2.

17. The method of claim 12, wherein the subject in need is one diagnosed with COVID-19 based on clinical symptoms of COVID-19 and a positive test for SARS-CoV-2.

18. The method of claim 17, wherein the method prevents disease progression in the subject.

19. A method for reducing cell to cell spread of a coronavirus in a subject in need thereof, the method comprising administering to the subject a composition comprising a serine protease inhibitor which irreversibly inhibits serine proteases and cannot diffuse across a phospholipid bilayer of a mammalian cell membrane.

20. A method for inhibiting dissemination of a coronavirus in a population of subjects, the method comprising administering to a plurality of subjects in the population a composition comprising a serine protease inhibitor which irreversibly inhibits serine proteases and cannot diffuse across a phospholipid bilayer of a mammalian cell membrane.

21. The method of claim 19, wherein the serine protease inhibitor is an irreversible inhibitor of transmembrane serine protease 2 (TMPRSS2).

22. The method of claim 21, wherein the serine protease inhibitor is alpha 1 antitrypsin (A1AT).

23. The method of claim 19, wherein the coronavirus is SARS-CoV-2.

24. A method for identifying an inhibitor of transmembrane serine protease 2 (TMPRSS2), the method comprising
contacting in vitro or ex vivo a plurality of recombinant cells with the test compound in the presence of a TMPRSS2 substrate, wherein the recombinant cells overexpress a proteolytically active recombinant transmembrane serine protease 2 (TMPRSS2), optionally comprising a peptide or protein tag element, and
detecting cleavage of the TMPRSS2 substrate, wherein reduced cleavage of the TMPRSS2 substrate in the presence of the test compound indicates the test compound is an inhibitor of TMPRSS2.

25. The method of claim 24, wherein the TMPRSS2 substrate is a flurogenic or colormetric substrate and cleavage is detected by detecting fluorescence or color, or a change in fluorescence or color.

26. The method of claim 25, wherein the substrate is Boc-Gln-Ala-Arg-7-Amino-4-methylcoumarin (BOC-QAR-AMC).

27. The method of claim 24, wherein the tag element is a peptide selected from a histidine tag, a FLAG tag, a human influenza hemagglutinin (HA) tag, a Myc tag, and a V5 tag.

28. The method of claim 24 wherein the tag element is a protein selected from a green fluorescent protein (GFP), GST glutathione-S-transferase (GST), β-galactosidase (β-GAL), luciferase, maltose binding protein (MBP), calmodulin binding protein (CBP), red fluorescence protein (RFP), and vesicular stomatitis virus glycoprotein (VSV-G).
